(19) United States
(12) Patent Application Publication
(10) Pub. No.: US 2003/0224407 A1
(43) Pub. Date: Dec. 4, 2003

(54) Methods for identifying the toxic/pathologic effect of environmental stimuli on gene transcription

(76) Inventors: Timothy Bertram, Old Lyme, CT (US); Michael J. Browne, Welwyn Garden City (GB); Peter Bugelski, Coventryville, PA (US); Paul England, St. Albans (GB); Ian Mitchell, Dorchester (GB); David Gwyn Morgan, Wayne, PA (US); Andrew Rut, Sunset Place (SG)

Correspondence Address: GLAXOSMITHKLINE, Corporate Intellectual Property - UW2220, P.O. Box 1539, King of Prussia, PA, 19406-0939, US

(21) Appl. No.: 10/383,212

(22) Filed: Mar. 6, 2003

Related U.S. Application Data
(62) Division of application No. 09/554,900, filed on Aug. 3, 2000, now abandoned.
Application No. 09/554,900 is a 371 of international application No. PCT/GB98/03445, filed on Nov. 20, 1998.
(60) Provisional application No. 60/066,241, filed on Nov. 20, 1997.

(51) Int. Cl. C12Q 1/68; G06F 19/00; G01N 33/48; G01N 33/50

(52) U.S. Cl. 435/6; 702/20

(57) ABSTRACT

Methods are disclosed for assessing the toxic or pathologic effects of a selected environmental stimulus or reagent on a mammalian cell by determining on a DNA grid a “fingerprint” hybridization pattern. The fingerprint pattern is characteristic of chemically or structurally diverse stimuli or reagents, which having a common adverse effect on gene transcription. A test compound is screened for a similar toxic effect by comparing its hybridization pattern on a similar grid to the fingerprint.

The present invention relates to the use of arrays or grids of mammalian gene sequence fragments from genomic (or cDNA) libraries for the screening of environmental factors, such as pharmaceutical compounds, physical factors, infectious agents, etc, for a toxic or pathologic effect upon gene transcription.

Mammalian cells frequently respond to exogenous stimuli of many types by altering the rate of transcription. For example, exposure of mammalian cells to environmental factors such as ultraviolet light, pharmaceutical compounds and many others can increase or decrease the quantity of messenger RNA produced by the cells. These changes in transcriptional regulation can result in toxic or pathological responses by the mammal. For example, where the external stimuli is prolonged exposure to UV rays, the toxic response of the mammal can be sunburn. Where the external stimuli is a compound known to be hepatotoxic, the response is liver damage. Where the external stimuli is a carcinogen, the toxic response is uncontrolled growth of cells.

The development of new pharmaceutical compositions and/or treatment regimens directed towards the treatment or prophylaxis of a variety of diseases, infectious or otherwise, relies quite heavily on the ability to screen candidate reagents for possible toxic or pathologic response. In normal drug development a novel chemical compound, novel biological composition, and the like is run through a battery of assays in vitro and in laboratory animals to ascertain its safety (i.e., lack of toxicity) and effectiveness.

The costs associated with the development of new pharmaceutical reagents are ever increasing, particularly when new compositions enter clinical trials. It is not unknown for promising pharmaceutical candidates to pass the appropriate laboratory tests and enter the expensive stage of animal and human clinical trials, only to present toxic or pathologic effects in the in vivo setting for the targeted mammalian patient, normally humans. The elimination of previously promising drug candidates at such a late stage in product development is a major factor in the high costs of new effective drugs which ultimately do pass the final clinical trials. Such late elimination of toxic compounds also results in unnecessary human suffering and wasted effort.

Methods have been described for obtaining information about gene expression and identity using so called “high density DNA arrays” or grids. See, e.g., M. Chee et al, Science, 274:610-614 (1996) and other references cited therein. Such gridding assays have been employed to identify certain novel gene sequences, referred to as Expressed Sequence Tags (EST) [Adams et al., Science, 252:1651-1656 (1991)]. A variety of techniques have also been described for identifying particular gene sequences on the basis of their gene products. For example, see International Patent Application No. WO91/07087, published May 30, 1991. In addition, methods have been described for the amplification of desired sequences. For example, see International Patent Application No. WO91/17271, published Nov. 14, 1991.

Accordingly, there exists a need for more efficient methods for screening novel pharmaceutical reagents, as well as other environmental stimuli or factors, to identify any toxic/pathogenic effect on gene transcription for both new drug development and new therapeutic regimens.

In one aspect, the invention provides a method of assessing the genetic effect of a selected environmental factor on a mammalian subject, said method comprising the steps of:

(a) providing a plurality of identical grids, each grid comprising a surface on which is immobilized at predefined regions on said surface a plurality of unique defined gene sequence fragments, said oligonucleotide sequences comprising genes or fragments of genes obtained from a healthy member of said mammalian species;

(b) exposing mammalian cells, tissue or organ to an environmental factor for a sufficient time to affect transcription of messenger RNA in said cells;

(c) extracting and isolating mRNA from said exposed cells, tissue or organ of step (b);

(d) extracting and isolating control mRNA from mammalian cells, tissue or organ not exposed to said factor;

(e) labelling the mRNA from steps (c) and (d);

(f) hybridizing the labeled mRNA from the exposed cells, tissue or organ to a first identical grid to produce a first hybridization pattern detectable by an increased quantity of fluorescence in contrast to the remainder of the grid;

(g) hybridizing the labeled control mRNA to a second identical grid to produce a second, control hybridization pattern; and

(h) comparing the first and second hybridization patterns to identify any change in said first pattern from the control pattern, indicative of an effect on transcriptional regulation of said mammalian cells, tissue or organ exposed to said factor.

The method of the invention thus employs the following steps. A plurality of identical DNA grids is prepared. At predefined regions on the grid surface, a plurality of defined amplified gene sequences (or oligonucleotide sequences) is immobilized. These gene sequences preferably are known or unknown genes, or fragments of genes, obtained from the cells (or a library of cells) of a healthy member of the mammalian species. Messenger RNA is isolated and extracted from mammalian cells which are not exposed to a selected environmental stimulus, thus forming the “control” RNA. The “test mRNA” is extracted from mammalian cells which have been exposed for a sufficient time to affect gene transcription to the selected stimulus. The control and test mRNA are randomly labeled, and each mRNA preparation is applied to an identical grid. The respective hybridization patterns are compared to identify any change in the test pattern from the control pattern, indicative of an effect on transcriptional regulation of the mammalian cells exposed to the stimulus. The determination of stimuli having a toxic or pathologic effect is useful, e.g., in the screening and development of new pharmaceutical agents and therapies.

The arrays or grids of mammalian gene sequence fragments from genomic (or cDNA) libraries used in the method of the invention may be high density DNA arrays or grids.

In another aspect, the method described above is performed for a “class” of stimuli, e.g., chemical or pharmaceutical compounds, which are to generate a common toxic or pathologic effect upon exposure to mammalian cells, e.g., hepatotoxicity. The method generates a “fingerprint” hybridization pattern for e.g., hepatotoxic, stimuli. Thus, test candidate drugs compositions may be screened for the likelihood of causing hepatotoxicity in mammalian cells by comparing the test hybridization pattern to the fingerprint at an early stage in drug development. Similarity between the fingerprint and the test pattern permit early elimination of the candidate drug from consideration, thus permitting only non-hepatotoxic compounds to proceed to drug development.

In still another aspect, the methods of the present invention may be performed to identify those genes which are the most responsive to a particular toxic effect of an external stimuli.

In still further aspects, the invention provides methods of identifying possible toxic or pathological effects of a variety of disparate physical stimuli, as well as chemical and pharmaceutical stimuli.

Other objects, features, advantages and aspects of the present invention will become apparent to those of skill in the art from the following description. It should be understood, however, that the following description, while indicating preferred embodiments of the invention, are given by way of illustration only. Various changes and modifications within the spirit and scope of the disclosed invention will become readily apparent to those skilled in the art from reading the following description and from reading the other parts of the present disclosure.

The present invention meets the needs of the art by providing a method of assessing the effect of any environmental factor or stimulus on gene expression in a mammalian subject by using DNA gridding techniques. Such techniques, employed as described below, permit the identification of genes which display a response to a test compound, permit the identification of a hybridization pattern characteristic of known physiologic effect in response to a test compound and permit the “fingerprinting” of certain selected toxic effects. The fingerprints are useful in screening new compounds or drug candidates for potential toxicity and in screening for the effect on gene transcription of other environmental stimuli. The information generated thereby can be used in the pharmaceutical industry to identify new drugs, in occupational safety evaluations of the workplace environment, and in many other industries and settings where it may be necessary to take measures to correct environmental stimuli which cause adverse effects in humans, and other mammals.

Several words and phrases used throughout this specification are defined as follows:

As used herein, the term “gene” refers to the genomic nucleotide sequence from which a cDNA sequence is derived. The term gene classically refers to the genomic sequence, which upon processing, can produce different RNAs.

By “gene product” it is meant any polypeptide sequence, peptide or protein, encoded by a gene. The term “genomic library” is meant to include, but is not limited to, plasmid libraries, PCR products from genomic libraries, cDNA libraries and known sequences. Methods for the construction of such libraries are well known by those skilled in the art. A genomic library may be adjusted to minimize the number of complete genes present in a single genomic insert to approximately one gene. Techniques for this adjustment are well known to the skilled artisan.

“Isolated” means altered “by the hand of man” from its natural state; i.e., that, if it occurs in nature, it has been changed or removed from its original environment, or both. For example, a polynucleotide or a polypeptide naturally present in a living animal in its natural state is not “isolated,” but the same polynucleotide or polypeptide separated from the coexisting materials of its natural state is “isolated,” as the term is employed herein. For example, with respect to polynucleotides, the term isolated means that it is separated from the chromosome and cell in which it naturally occurs.

“Pathogenic effect” or “pathologic effect”, as used herein, refers to a change in gene expression which may cause a disease or disorder. The change is due to exposure of a mammal or mammalian cell to some environmental stimulus, as detailed below.

As used herein, the term “solid support” refers to any substrate which is useful for the immobilization of a plurality of defined materials derived from a genomic library by any available method to enable detectable hybridization of the immobilized polynucleotide sequences with other polynucleotides in the sample. Among a number of available solid supports, one desirable example is the support described in International Patent Application No. WO91/07087, published May 30, 1991. Examples of other useful supports include, but are not limited to, nitrocellulose, nylon, glass, silica and Pall BIODYNE C membrane. It is also anticipated that improvements yet to be made to conventional solid supports may also be employed in this invention.

The term “grid” means any generally two-dimensional structure on a solid support to which the defined materials of a genomic library are attached or immobilized. Preferably according to this invention, three types of grids are useful. One grid useful in this invention contains as its defined oligonucleotide materials, unique nucleic acid sequences [or “tags”; or expressed sequence tags (“EST”)] from all human genes identified. A second useful grid contains unique nucleic acid ESTs from genes cloned from a tissue or a cell line. Still a third type of grid useful in the present invention contains unique nucleic acid tags from genes classified as particularly relevant to identification of a selected environmental toxicity. Grids are desirably constructed from animal species used in the preclinical assessment of compound safety.

As used herein, the term “predefined region” refers to a localized area on a surface of a solid support on which is immobilized one or multiple copies of a particular amplified gene region or sequence and which enables hybridization of that clone at the position, if hybridization of that clone to a sample polynucleotide occurs.

By “immobilized,” it is meant to refer to the attachment of the genes to the solid support. Means of immobilization are known and conventional to those of skill in the art, and may depend on the type of support being used.

The terms “environmental factor” or “environmental stimuli” are used herein to describe a wide variety of physical, chemical or biological factors which cause changes in gene transcription in a mammalian cell when the mammal itself, or a culture of such mammalian cells, is exposed to that factor. For example, physical environmental stimuli can include, without limitation, the diet of the mammal, an increase or decrease in temperature; an increase or decrease in exposure to ionizing or ultraviolet radiation, and the like. A biological/chemical stimuli can include, without limitation, administering a transgene to the mammal, or eliminating a gene from the mammal; administering an exogenous synthetic compound or exogenous agent or an endogenous compound, agent or analog thereof to the mammal.

As an example, an exogenous synthetic compound can be a pharmaceutical compound, a toxic compound, a protein, a peptide, a chemical composition, among other. An exogenous agent can include natural pathogens, such as microbial agents, which can alter gene transcription. Examples of pathogens include bacteria, viruses, and lower eukaryotic cells such as fungi, yeast, molds and simple multicellular organisms, which are capable of infecting a mammal and replicating its nucleic acid sequences in the cells or tissue of that mammal. Such a pathogen is generally associated with a disease condition in the infected mammal.

An endogenous compound is a compound which occurs naturally in the body. Examples include hormones, enzymes, receptors, ligands, and the like. An analogue is an endogenous compound which is preferably produced by recombinant techniques and which differs from said naturally occurring endogenous compound in some way.

By “transcriptional effect” is meant an increase or decrease in rate of transcription in the mammalian cells exposed to the stimuli.

A “fingerprint” as used herein is defined as a characteristic hybridization pattern on a grid indicating a common toxicological response, i.e., similar increases in gene transcription that result in similar tissue damage. For example, using the methods described herein, one may generate a “hepatotoxic” fingerprint, which can be used to identify compounds which are likely to have a toxic effect on the liver, and so on.

By “label” as used herein is meant any conventional molecule which can be readily attached to mRNA and which can produce a detectable signal, the intensity of which indicates the relative amount of hybridization of the mRNA to the DNA fragment (oligonucleotide) on the grid. Preferred labels are fluorescent molecules or radioactive molecules. A variety of well-known labels can be used.

### Method of the Invention

A. The Grids

According to the present invention, a method is provided which enables the association of selected environmental stimuli with changes in gene transcription. One of the specific applications of this technology is the understanding and prediction of toxic reactions to environmental manipulations and modifications, such as those stimuli listed above. Another application is in pre-clinical and clinical drug development, where the method of this invention enables the screening of compounds having a similar toxic effect on gene transcription by comparison to the effect of another stimulus.

In the practice of this method, a plurality of identical grids is prepared, so that each grid carries on its solid surface a plurality of defined unique gene (oligonucleotide) sequences immobilized at predefined regions on the surface. The gene sequences immobilized on the grids are as defined above, i.e., as unique nucleic acid tags from all human or other mammalian genes, or from only a selected tissue, e.g., reticulocytes, or the liver, or a selected cell line, or from genes known to be relevant to environmental toxicity, e.g., the lung, kidney, heart, blood cells, etc. These genes or fragments of genes immobilized on the grids may be obtained from an oligonucleotide library of a healthy member of the mammalian species, e.g., a healthy human. Other mammals of interest include, without limitation, a non-human primate, a rodent, and a canine.

For the purposes of this invention, it is not necessary that the grids reflect a single target organ, although such a specific target grid can be used. It is anticipated that the response of the mammalian cell to various environmental stimuli that effect gene transcription is likely to be stereotypic of genes in other cells. Thus, the grid can be prepared from red or white blood cells, reticulocytes, or undifferentiated cells, even where the particular toxicological effect is damage to the liver or some other particular tissue. Alternatively, such a grid can be prepared from hepatocytes only, or from cells from the effected organ or tissue only. All grids are anticipated to reflect the same hybridization pattern upon exposure to a reagent or stimulus that is known as hepatotoxic. The same is true regardless of the type of toxicological damage, e.g., cardiac damage, kidney damage, hematopoietic cell damage, etc.

The gene fragments immobilized on the grid may be obtained from a random cDNA library of the target mammal using known techniques. Alternatively, a cDNA library of genes from a selected organ or tissue may be prepared as the source of the sequences immobilized on the grid. The RNA is isolated and reverse transcribed to cDNA using standard procedures for molecular biology such as those disclosed by Sambrook et al., MOLECULAR CLONING, A LABORATORY MANUAL, 2nd Ed; Cold Spring Harbor Laboratory Press, Cold Spring Harbor Lab Press, Cold Spring Harbor, N.Y. 1989. The cDNA library is then constructed in accordance with procedures described by Fleischmann et al. Science, 1995, 269:496-512. For the purposes of the present invention, a cDNA library can comprise a plasmid library, PCR products from a cDNA library, or known sequences.

A plurality of genes or gene fragments, whether known or random and unknown, from the selected library are gridded onto a surface of a solid support at predefined locations or regions, preferably at 6× coverage. By “plurality of materials derived from the genomic library” it is meant to include, but is not limited to, individual clones spotted onto and grown on a surface of the solid support at predefined locations or regions; or plasmid clones isolated from said library, PCR products derived from the plasmid clones, or oligonucleotides derived from sequencing of the plasmid clones, which are immobilized to the surface of the solid support at predefined locations or regions. As selection of genes involved in e.g., carcinogenicity, apoptosis, inflammation, metabolism of compounds etc, may be used.

The grids used in the invention may contain, e.g., up to 5,000 genes or gene fragments. The grids preferably contain up to 1,500 genes or gene fragments, e.g., 100 to 1,500 genes or gene fragments, more preferably about 1,000 genes or gene fragments.

Numerous conventional methods are employed for immobilizing these gene sequences (oligonucleotides) to surfaces of a variety of solid supports. See, e.g., Affinity Techniques, Enzyme Purification: Part P, Methods in Enzymology, Vol. 34, ed. W. B. Jakoby, M. Wilcheck, Acad. Press, NY (1971); Immobilized Biochemicals and Affinity Chromatography, Advances in Experimental Medicine and Biology, Vol. 42, ed. R. Dunlap, Plenum Press, NY (1974); U.S. Pat. No. 4,762,881; U.S. Pat. No. 4,542,102; European Patent Publication No. 391,608 (Oct. 10, 1990); or U.S. Pat. No. 4,992,127 (Nov. 21, 1989).

One desirable method for attaching these materials to a solid support is described in International Application No. PCT/US90/06607 (published May 30, 1991). Briefly, this method involves forming predefined regions on a surface of a solid support, where the predefined regions are capable of immobilizing the materials. The method makes use of binding substrates attached to the surface which enable selective activation of the predefined regions. Upon activation, these binding substances become capable of binding and immobilizing the materials derived from the genomic library.

Any of the known solid substrates suitable for binding nucleotide sequences at predefined regions on the surface thereof for hybridization and methods for attaching nucleotide sequences thereto may be employed by one of skill in the art according to the invention.

As described above the genes or gene fragments may be of known or unknown function. In a fingerprinting method it is not necessary to know the function of every gene since the method may not be looking at specific pathways of toxicity but at distinct patterns of gene expression in response to environmental factors.

B. Obtaining the mRNA for Hybridization to the Grids

The selected mammalian cells, tissues or organs to be examined for transcription changes are subjected to the environmental stimulus for a sufficient time to affect transcription of messenger RNA in the cells. This “exposing” step can occur by treating or exposing a living healthy animal or human to the stimulus. For example, the selected mammal may be administered a reagent, such as an exogenous or endogenous compounds as described above. Alternatively, the mammal may be exposed to a physical stimulus, e.g., UV radiation.

Alternatively, a mammalian cell culture or tissue culture, or viable organ, e.g., liver, heart, etc., may be exposed to the stimulus in vitro. A control mRNA source is an untreated animal, tissue, organ or cell culture.

The exposure to the environmental stimulus, which may be stimuli known to cause a specific physical effect, e.g., hepatocyte damage, cancer, etc., occurs for a time sufficient to result in the alteration from the normal of the transcription level of the cells so exposed. The sufficient time will depend upon the particular stimulus being studied and, in fact, determination of a sufficient stimulus time is well within the skill of the art.

Where the mRNA source is a cell culture, the culture is then incubated under a selected set of defined in vitro or in vivo conditions to produce a test culture. In addition, non-exposed cells are also cultured under the same set of defined conditions to produce a control culture. By “defined conditions” it is meant, but is not limited to, standard in vitro culture conditions recognized as normal (i.e., non-pathogenic) for a selected mammalian cell, as well as in vitro conditions which reflect or mimic in vivo pathogenic settings (conditions) such as heat shock, auxotrophic, osmotic shock, antibiotic or drug selection/addition varied carbon sources, and aerobic or anaerobic conditions, and in vivo, pathogenic conditions. Preferably, such conditions are predetermined to allow maximum growth of the non-exposed cells.

The cells are then harvested from the animal, organ, tissue or cell culture by conventional means. Harvesting can be performed during various growth stages of the cells to ascertain the essentiality of a particular gene during different stages of growth. For example, harvesting can be performed during early logarithmic growth, late logarithmic growth, stationary phase growth or late stationary growth. RNA (or DNA) is then extracted and isolated from the harvested non-exposed cells of the control culture, and RNA is extracted and isolated from the cells exposed to the stimulus of the test culture using standard methodologies well known to those skilled in the art.

mRNA extracted from the cells of the control culture and from the cells of the test culture are then used to generate labeled probes. When mRNA from the control and test cells is used to generate the probes, isolated mRNA is labeled according to standard methods using random primers, preferably hexamers, and reverse transcriptase. Such methods are routinely performed by those skilled in the art. All mRNA from the “control” or the “exposed” source is randomly labeled by conventional means, such as nick translation, multiprime labelling or other commonly used enzymatic labeling methodology. Known conventional methods for labelling the mRNA sequences may be used and make hybridization of the immobilized materials detectable. For example, fluorescence, radioactivity, photoactivation, biotinylation, energy transfer, solid state circuitry, and the like may be used in this invention.

C. Hybridization to the Grids

These labeled mRNAs are then used as hybridization probes against the identical high density grids. Labeled probes prepared from mRNA extracted from the test culture are hybridized to one grid to produce a “test” hybridization pattern. Labeled probes from the mRNA extracted from the cells of the control culture are hybridized to a second identical grid, resulting in a “control” hybridization pattern.

The generated test hybridization patterns and control hybridization patterns are then compared. In the control pattern, the mRNA binds to certain genes or gene fragments in the grid in proportion to the expression of the mRNA of such genes in a normal cell. The pattern is detectable by an increased quantity of detectable signal, e.g., fluorescence, at locations on the grid of those genes which are normally expressed in greater quantities that others in the remainder of the grid.

In the test grid, genes for which transcription is enhanced by the stimulus will be bound by a greater amount of labeled mRNA, and genes for which transcription is reduced by the stimulus will be bound by a lesser amount of labeled mRNA, thus altering the hybridization pattern from that of the control. Comparison of the test and control patterns reveals the effect of the test compound on transcription of certain genes located at the predefined locations on other grid.

D. The Fingerprints

Thus, where the test compound or stimulus is a stimulus known to cause a physiological effect, for example, a toxic reaction of a subject resulting in damage to a major organ, e.g., liver, kidney, heart, blood cells, the method of this invention may be performed to provide a hybridization pattern which correlates with that damage. Most desirably, for preclinical drug screening according to this invention, any collection of known and structurally distinct toxicants which have the same physiological effects, e.g., hepatotoxicity, can be employed in this method to generate a characteristic “fingerprint” hybridization pattern for hepatotoxic stimuli.

Where it is desired to produce a common hybridization pattern such known toxicants, a set of grids are calibrated with a repertoire of the structurally diverse toxicants that produce the same pathological/toxicological reaction; e.g. hepatotoxicity or nephrotoxicity. In other words, labeled RNA from a mammalian cell source exposed to the known toxicants are hybridized to identical grids to produce a common toxicant hybridization pattern. If the variety of known toxicants produce a characteristic common hybridization pattern, the common toxicological responses are likely to be the result of similar increases in transcription of selected genes, resulting in similar tissue damage. This toxicological fingerprint pattern may be used along with the “control” pattern for comparison with the pattern of a test compound/stimulus of unknown function or result. Thus the common fingerprint for, e.g., hepatotoxicity, is used to screen a stimulus of unknown function or effect to determine if that stimulus is likely to produce hepatotoxicity in the mammal.

Similarity in the “test” pattern to the hepatotoxic fingerprint enables the putative identification of the test compound as a hepatotoxic compound. Thus, if the test compound was a drug candidate, it can be eliminated from consideration at the earliest stages of drug development on the basis of its effects on gene transcription as measured on the grids. Similarly the method permits the test compound or stimulus, if an environmental factor present in e.g., the workplace, such as radiation, etc., to be identified as a potential health hazard, and corrected.

According to this method, therefore, a battery of fingerprint hybridization patterns may be prepared for all known toxicants. Any new drug candidate or other environmental stimulus may be screened by the above method for probable toxicological effects by comparison to standard fingerprints for other known stimuli causing liver damage, kidney damage, damage to the hematopoietic systems, etc. Such a screening method will enable quick and early evaluation of environmental stimuli, particularly new drug candidates.

Fingerprint hybridization patterns may be stored in a database and pattern matching performed by datamining.

E. Preclinical Embodiments of the Method

In a particularly desirably embodiment of the method of this invention, in vitro effects of pharmacologically relevant concentrations of compounds on gene expression in blood cells are examined using the methods of this invention. A gene expression fingerprint is developed through this methodology by exposing the nucleated blood cells, e.g., reticulocytes, white cells, to a variety of toxicants as described above. The resulting fingerprint is used subsequently to predict whether a novel compound is likely to also produce a similar pathological reaction. The information assists decisions about which compounds to take forward to clinical development, and enhances safety in the clinic through accurate and early prediction of toxicity.

An alternative embodiment of the method of this invention is to analyze the in vitro effects of pharmacologically relevant concentrations of compounds on gene expression in blood cells.

The Genes and Proteins Identified by the Method:

In still another embodiment, the method described above, and/or the fingerprints generated for certain selected toxicities may be useful in identifying novel genes that may have a significant impact on the compound's toxicity. Application of the compositions and methods of this invention as above described also provides other compositions, such as any isolated gene sequence which is unusually reactive to the toxic result of one or more known toxicants.

For example, in a desirable embodiment, the methods of this invention is useful in a clinical setting. Gene expression grids may aid in the identification of the mechanism underlying the occurrence of pathological reactions and toxicity in a minority of patients during human trials. Using human grids, gene expression in cells derived from patients/volunteers known to have experienced the adverse event in question during a clinical trial can be compared to gene expression from those who remained well. Ideally as described above, mRNA is obtained from cells of the target organ, but may also include mRNA obtained from blood cells in which transcription can be altered, e.g., white blood cells. By comparing hybridization patterns for the affected patients vs. the well patients, a defined genetic fingerprint or genes that are differentially expressed to a significant degree may be obtained.

An embodiment of the invention is any gene sequence identified by the methods described therein. These gene sequences associated with the toxic reaction are used to obtain full-length cDNA clones by conventional methods. The genes may be studied in greater detail; e.g. through sequencing and mutation analysis.

These gene sequences may be employed in conventional methods to produce isolated proteins encoded thereby. To produce a protein of this invention, the DNA sequences of a desired gene invention or portions thereof identified by use of the methods of this invention are inserted into a suitable expression system. In a preferred embodiment, a recombinant molecule or vector is constructed in which the polynucleotide sequence encoding the protein is operably linked to a heterologous expression control sequence permitting expression of the human protein. Numerous types of appropriate expression vectors and host cell systems are known in the art for mammalian (including human), insect, yeast, fungal and bacterial expression.

The transfection of these vectors into appropriate host cells, whether mammalian, bacterial, fungal or insect, or into appropriate viruses, results in expression of the selected proteins. Suitable host cells, cell lines for transfection and viruses, as well as methods for construction and transfection of such host cells and viruses are well-known. Suitable methods for transfection, culture, amplification, screening and product production and purification are also known in the art.

In one embodiment, the essential genes and proteins encoded thereby which have been identified by this invention can be employed as diagnostic compositions useful in the diagnosis of a disease or infection by conventional diagnostic assays. For example, a diagnostic reagent can be developed which detectably targets a gene sequence or protein of this invention in a biological sample of an animal. Such a reagent may be a complementary nucleotide sequence, an antibody (monoclonal, recombinant or polyclonal), or a chemically derived agonist or antagonist. Alternatively, the essential genes of this invention and proteins encoded thereby, fragments of the same, or complementary sequences thereto, may themselves be used as diagnostic reagents. These reagents may optionally be detectably labeled, for example, with a radioisotope or calorimetric enzyme. Selection of an appropriate diagnostic assay format and detection system is within the skill of the art and may readily be chosen without requiring additional explanation by resort to the wealth of art in the diagnostic area.

Additionally, genes and proteins identified according to this invention may be used therapeutically. For example, genes identified as essential in accordance with this method and proteins encoded thereby may serve as targets for the screening and development of natural or synthetic chemical compounds which have utility as therapeutic drugs for the treatment of disease states associated with exposure to environmental stimuli. As an example, a compound capable of binding to a protein encoded by an essential gene thus preventing its biological activity may be useful as a drug component preventing diseases or disorders resulting from exposure of the mammalian cells to the environmental stimuli. Alternatively, compounds which inhibit expression of an essential gene are also believed to be useful therapeutically. In addition, compounds which enhance the expression of genes essential to the growth of an organism may also be used to promote the growth of a particular organism.

Conventional assays and techniques may be used for screening and development of such drugs. For example, a method for identifying compounds which specifically bind to or inhibit proteins encoded by these gene sequences can include simply the steps of contacting a selected protein or gene product with a test compound to permit binding of the test compound to the protein; and determining the amount of test compound, if any, which is bound to the protein. Such a method may involve the incubation of the test compound and the protein immobilized on a solid support. Still other conventional methods of drug screening can involve employing a suitable computer program to determine compounds having similar or complementary structure to that of the gene product or portions thereof and screening those compounds for competitive binding to the protein. Identical compounds may be incorporated into an appropriate therapeutic formulation, alone or in combination with other active ingredients. Methods of formulating therapeutic compositions, as well as suitable pharmaceutical carriers, and the like are well known to those of skill in the art.

Accordingly, through use of such methods, the present invention is believed to provide compounds capable of interacting with these genes, or encoded proteins or fragments thereof, and either enhancing or decreasing the biological activity, as desired. Thus, these compounds are also encompassed by this invention.

All publications, including but not limited to patents and patent applications, cited in this specification are herein incorporated by reference as if each individual publication were specifically and individually indicated to be incorporated by reference herein as though fully set forth.

Numerous modifications and variations of the present invention are included in the above-identified specification and are expected to be obvious to one of skill in the art. Such modifications and alterations to the compositions and processes of the present invention are believed to be encompassed in the scope of the claims appended hereto.

The invention is illustrated by the following examples.

### EXAMPLES

Gene Expression Measurements using Microarrays

Source of Cloned Sequences

Sequences were derived from several sources. IMAGE clones (human derived cDNA sequences inserted into bacterial plasmids) were ordered from Research Genetics in duplicate. The stocks were streaked out onto agar plates, and 3 colonies per clone were PCR screened with gene specific primers to determine which clones contained the correct sequences. Positive clones were then sequenced (ABI automated sequencer) and checked against the sequence database to ensure the clones were correct. Six clones were prepared de novo by PCR from SB human cDNA. Rat, mouse and dog clones were prepared de novo by Reverse Transcriptase-PCR (RT-PCR) from species specific RNAs using gene specific primers and were also sequence confirmed. Stocks containing the correct clones were preserved as glycerol stocks. In total the microarray comprises of: 77 sequences representing 45 different mammalian genes; and 5 yeast gene sequences.

Preparation of DNA for the Microarray

DNA was amplified in 96 well plates on a Perkin Elmer 9600 Thermal Cycler using a mixture of vector primers specific for BSK and pT7T3 (Pharmacia). Total reaction volume was 100 ul containing the following: 1 ul of culture from the stock containing the correct clone, 10 ul 10×PCR buffer (10×=300 mM Tricine, 20 mM Magnesium Chloride, 50 mM BetaMercaptoEthanol), 0.5 ul Perkin Elmer Taq polymerase (5U/ul), 200 uM dNTP's (Amersham), 50 ng each primer, including Universal Forward and Reverse, as well as 2 primers made to the Pharmacia pT7T3 vector. 38 amplification cycles were carried out: 2 minutes @94° C. initial soak (1 cycle); 35 seconds @ 94° C. (autoincrement 1 sec per cycle); 30 seconds @ 55° C.; 1 minute 45 seconds @72° C. (autoincrement 1 sec per cycle) and a 10 minutes @ 72° C. final extension period.

PCR yields and specificity were checked by agarose gels, and the products were Ethanol precipitated as follows, in Nunc 96 well V-bottom plates. 10 ul of 3M Sodium Acetate was added to the 100 ul PCR reaction, mixed, then 275 ul of 100% Ethanol was added, and mixed again. Plates were stored at −20° C. for 20 minutes, followed by a 30 minute spin in a Beckman GS-6R tabletop centrifuge using Beckman Microplus carriers, at 3000 rpm, 4° C. Pellets were visible at the bottom of the wells, which were washed with 50 ul 70% Ethanol, and spun again at 3000 rpm for 20 minutes. Pellets were air dried, and resuspended at 300 ng/ul in distilled water.

Preparation of the Microarray

A 10 ul aliquot from each of the suspended PCR products was mixed with an equal volume of 11M NaSCN (J. T. Baker) and deposited into individual wells of 96-well microtiter plates (Nunc). Approximately 1 nl of each sample was arrayed in duplicate onto silanized (3-aminopropyl trimethoxy silane treated) glass slides using high-speed robotics (Molecular Dynamics Generation II Microarray System). The average diameter of each array element was measured at 215 microns with the spot-to-spot centers at a distance of 500 microns. After printing, the slides were allowed to air dry and then placed into a vacuum oven for 2 hours at 80° C. Prior to hybridization, the slides were washed for 10 minutes in isopropanol, boiled for 5 minutes in ddH₂O, and air dried.

Preparation of cDNA Probes

Probes were prepared by simultaneous reverse transcription and labelling in the presence of a fluorophore. The reactions were carried out with a Gibco-BRL Superscript II™ kit (Preamplification System for First Strand cDNA Synthesis) and the protocol was as follows:

10 ug of Quiagen cleaned sample RNA was mixed with 2 ug of anchored oligo dT₂₀ (Cambio) in DEPC treated water to a final volume of 11.2 ul. The mix was heated to 68° C. for 10 minutes and returned to ice for 1 minute.

A PCR reaction mix was prepared and kept on ice until required: 2 ul×10 PCR buffer (supplied with kit), 2 ul 25 mM MgCl₂, 1 ul dNTP mix (to give 500 uM final concentration of each of dATP, dGTP and dTTP, and a final concentration of 280 uM of dCTP), 0.8 ul Cy3™ dCTP (Amersham) to give a final concentration of 40 uM and 2 ul 0.1M DTT to give a total volume of 7.8 ul.

The annealed RNA (11.2 ul) was added, on ice, to the 7.8 ul PCR reaction mix, mixed gently and then incubated at 39.5° C. for 5 minutes. 1 ul of Superscript II™ (200U/ul) was added, mixed gently, and the mix incubated at 39.5° C. for a further 60 minutes. A further 1 ul of Superscript II™ was added and incubated at 39.5° C. for another 60 minutes. The reaction was terminated by heat inactivating the Superscript II at 68° C. for 5 minutes.

RnaseH (2U/ul) was added and incubated at 39.5° C. for 20 minutes and the probe cleaned up by running through a Quiaquick™ PCR column according to the manufacturers instructions.

Yeast control RNA's were made by in vitro transcription of cloned YGL097, YDR432, YML113, YFL021 and YGR014 cDNA's using a Riboprobe in vitro Transcription System (Promega). For quality assurance purposes, the yeast RNA's were added to the reaction at ratio's of 1:100, 1:1,000, 1:5,000, 1:10,000 and 1:20,000 (wt/wt) respectively. After incubating the reaction at 39.5° C. for 60 minutes, an additional 1 ul of Superscript 11 RT was added and incubated at 39.5° C. for a further 120 minutes. Following termination of the reaction, 1 ul of RNase A (10 ug/ul) and 1 ul of RNase H were added and incubated at 39.5° C. for 20 minutes. Unincorporated label was removed by passing the reaction down a Qiaquick PCR Purification Kit (Qiagen) according to the manufacturers protocol. To ensure the probe was completely free of unincorporated nucleotide, the above procedure was repeated before drying the probe to completion in vacuo.

Hybridisation

The probe was dried down and resuspended in 12 ul (for full-length cover slips) or 4 ul (for small cover slips) of hybridisation buffer (5×SSC, 0.1% SDS, 0.25 uM pA₂₀) and incubated at 100° C. for 5 minutes. The probe mixture was pipetted onto the microarray surface and covered with a glass cover slip and sealed with latex glue. The microarray was transferred to a hybridisation oven and incubated at 42° C. for 15 hours.

Washing

The glue and coverslip was removed whilst the microarray slide was immersed in a bath of low stringency buffer (2×SSC, 0.1% SDS) at room temperature and the slide incubated for 5 minutes. The slide was then washed in a high stringency wash (0.5×SSC, 0.1% SDS) on a flat bed shaker at room temperature for 5 minutes. After repeating the high stringency wash, the microarray slide was quicky placed in a 50 ml Falcon tube and centrifuged (2 minutes at 200×g) to remove any traces of wash buffer.

Data Capture

Fluorescence from the microarray was detected and quantitated using a Molecular Dynamics Gen II scanner. The fluorescent signal is measured as intensity per mm². A background measurement for each spot was taken in an area surrounding each spot.

Analysis of Data

Gene Expression Analysis from Microarrays

After background subtraction the density for each spot was “normalised” by calculating the ratio of the spot density to the sum of all the spot densities and expressed as the nDxA (for normalised density per unit area). The ratio (T/C) of the treated vs control values was calculated for each spot for each treatment and time point. This was done for spot set 1 and spot set 2 separately. Starting with spot set 1 sequences having T/C ratios of >2 and <0.5 were identified as showing differential gene expression. If the signal was weak (<0.35) in both spot sets for both treated and control samples, that sample was removed from the analysis as being outside the detectable range. The spot images of each of the identified sequences were examined for dust spots or other “noise” which would give an incorrect densitometric value. Each differentially expressed sequence was ranked according to fold increase/decrease.

1. A method for identifying an effect on transcriptional regulation underlying the occurrence of a pathological reaction to a pharmaceutically active substance, said method comprises the steps of;
(a) providing a plurality of identical grids, each grid comprising a surface on which is immobilized at predefined regions on said surface a plurality of defined unique oligonucleotide sequences, wherein said sequences comprise a gene or gene fragment obtained from a healthy human;
(b) exposing human cells, tissue or organ obtained from a human not having said pathological reaction to said pharmaceutically active substance for a sufficient time to affect transcription of mRNA in said cells, tissue or organ;
(c) extracting, isolating and labeling mRNA from said exposed cells, tissue or organ of step (b);
(d) exposing human cells, tissue or organ obtained from a human having said pathological reaction to said pharmaceutically active substance for a sufficient time to affect transcription of mRNA in said cells, tissue or organ;
(e) extracting, isolating and labeling mRNA from said cells, tissue or organ of step (d);
(f) hybridizing the mRNA of step (c) to a first identical grid to produce a first hybridization pattern;
(g) hybridizing the mRNA of step (e) to a second identical grid to produce a second hybridization pattern;
(h) comparing the first pattern of step (f) and the second pattern of step (g) to identify any difference between said first pattern and second pattern, said difference being indicative of an effect on transcriptional regulation of said cells, tissue or organ in said human having said pathological reaction.

2. The method of claim 1 wherein said grid further comprises unique nucleic acid tags from genes cloned from selected tissue or cell line.

3. The method of claim 1 wherein said defined unique oligonucleotides are from genes that are particularly relevant to the identification of a selected toxicity.

4. The method of claim 2 wherein said unique nucleic acid tags are from genes that are particularly relevant to the identification of a selected toxicity.

5. The method of claim 1 further comprising the step of identifying said gene or genes differentially expressed in said human cells, tissue or organ obtained from said human having said pathological reaction as compared to genes expressed in said human cells, tissues or organ obtained from said human not having said pathological reaction.

6. The method of claim 5 further comprising the step of expressing said protein or proteins encoded by said identified gene or genes.

7. The method of claim 6 wherein said step of expressing comprises inserting said identified gene into an expression vector.

8. The method of claim 6 further comprising the step of identifying said protein.

9. The method of claim 7 further comprising the step of identifying said protein.
